(19) United States
(12) Patent Application Publication
(10) Pub. No.: US 2012/0172798 A1
(43) Pub. Date: Jul. 5, 2012

(54) DEVICES AND METHODS FOR ACCESSING A CEREBRAL VESSEL

(76) Inventors: John Miller, Redwood City, CA (US); Scott Wilson, Redwood City, CA (US); Down Davilla, Sunnyvale, CA (US)

(21) Appl. No.: 13/315,283

(22) Filed: Dec. 8, 2011

Related U.S. Application Data
(63) Continuation-in-part of application No. 12/386,506, filed on Apr. 17, 2009.
Application No. 12/386,506 is a continuation-in-part of application No. 11/490,843, filed on Jul. 21, 2006, now abandoned.

(51) Int. Cl. A61M 25/10

(52) U.S. Cl. 604/103.09; 604/96.01

(57) ABSTRACT

The present invention is directed to methods and devices for accessing a cerebral vessel. The system includes a support catheter having a balloon, which can be advanced into small and tortuous vessels. By advancing the support catheter nearer to the obstruction then can be achieved with conventional guide catheters, the support catheter reduces the likelihood vessel compression and collapse when manipulating a working catheter. The balloon may be used to temporarily stop blood flow.

### CROSS-REFERENCE TO RELATED APPLICATIONS

The present application a continuation in part of U.S. patent application Ser. No. 12/386506, filed Apr. 17, 2009, which is a continuation in part of U.S. patent application Ser. No. 11/490,843, filed Jul. 21, 2006, which are incorporated herein by reference.

### BACKGROUND OF THE INVENTION

The present invention is directed to devices and methods for removing obstructions from a cerebral vessel. In particular, the present invention may be useful in removing obstructions from the M1 or M2 Middle Cerebral Artery (MCA). Of course, the present invention may find uses in other parts of the cerebral vasculature and in other parts of the body.

Various problems can occur when attempting to remove hard, well organized and/or impacted clots or obstructions with a retrieval device in the Middle Cerebral Artery. Vessel compression and displacement can occur in the soft, flexible and relatively unsupported cerebral vasculature when force is applied to the clot and the retriever to dislodge the clot. For example, the proximal portion of the MCA and/or ICA can be particularly susceptible to vessel compression and collapse which may cause the vessel to fold up on itself making successful retraction difficult. Vessel compression and displacement can also produce excessive frictional forces between the vessel and the retriever.

Another problem, which can occur, is that the force exerted on an obstruction to dislodge and remove the obstruction may be exerted in a direction, which is skewed relative to the longitudinal axis of the vessel. This problem may be particularly troublesome when the obstruction is in the MCA. The MCA arises from the top of the ICA and travels in a direction roughly 90 degrees from the ICA. The tortuous vasculature often results in the application of force to the obstruction, which is not aligned with the axis of the vessel.

### SUMMARY OF THE INVENTION

In one aspect of the present invention, a support catheter is provided which can be advanced over an obstruction retrieval device and guided into the proximal portion of the MCA. Positioning the support catheter in the MCA at a position close to the retriever can provide a more effective transmission of force to the retriever. Positioning the support catheter in this manner may also provide a more axially oriented force than would be provided if the support catheter were not able to navigate to the MCA. Typical guide catheters, for example, are too stiff to navigate the tight bends and tortuosity of the distal ICA and the siphon. The support catheter of the present invention, on the other hand, is flexible enough to navigate the distal ICA and the siphon. The support catheter may also be large enough to allow for a microcatheter to be positioned between the support catheter and the retriever. The proximal portion of the support catheter may be somewhat stiff and rigid for optimized advancement and support.

The present invention is also directed to systems and methods for removing obstructions. The system may include a guide catheter having a lumen coupled to a suction source to aspirate the obstruction as is known. A microcatheter is positioned within the lumen of the guide catheter. The microcatheter is used to introduce the obstruction retriever into the obstruction. A support catheter is also provided which is advanceable over the retriever and optionally over the microcatheter as well. A distal end of the guide catheter is positioned proximal to the MCA. The support catheter may be advanced with the microcatheter and/or retriever as a system or may be introduced before introduction of the microcatheter and/or retriever.

The microcatheter is advanced through the guide catheter and into or distal to the obstruction. Withdrawal of the microcatheter exposes a portion of the retriever, which is now in contact with the obstruction. If the retriever is deployed distal to the obstruction, the microcatheter and retriever are withdrawn together until the retriever engages the obstruction. The end of the support catheter is advanced to a position near the obstruction to provide the advantages described herein. The retriever may then be pulled proximally to dislodge and remove the obstruction. By advancing the support catheter nearer to the obstruction and retriever than would be possible with typical guide catheters, the problems concerning vessel compression and collapse discussed above may be reduced or avoided since the support catheter will provide a more axially directed force relative to the vessel. Another advantage of positioning the support catheter just proximal to the obstruction is that suction can be applied to the lumen of the support catheter to assist in removal of the obstruction.

In another aspect of the present invention, the support catheter may also include a balloon to temporarily stop blood flow. The balloon may be positioned 5-15 cm from the distal end of the support catheter so that the distal portion of the support catheter remains flexible to navigate into distal and/or tortuous vessels such as the MCA.

These and other aspects of the present invention will become apparent from the following description. The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 shows a system for removing an obstruction from a vascular location.

FIG. 2 shows a retriever, microcatheter, support catheter and guide catheter in accordance with the present invention.

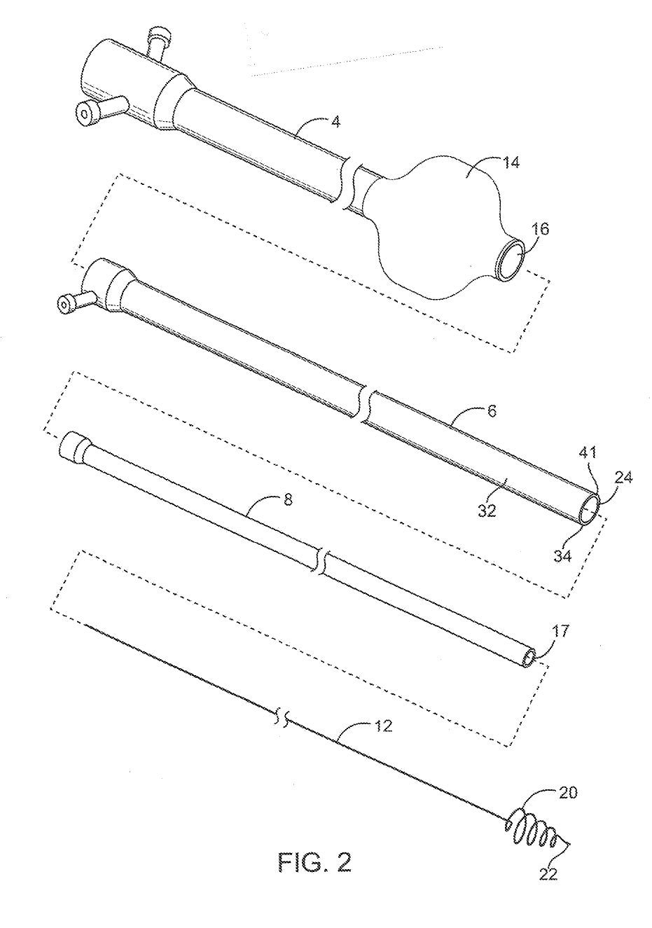

FIG. 3 shows a cross-sectional view of the construction of the support catheter.

FIG. 4 shows removal of an obstruction in accordance with the present invention.

FIG. 5 shows a cross-sectional view of another support catheter.

FIG. 6 shows another support catheter having a balloon to occlude blood flow.

### DESCRIPTION OF THE PREFERRED EMBODIMENTS

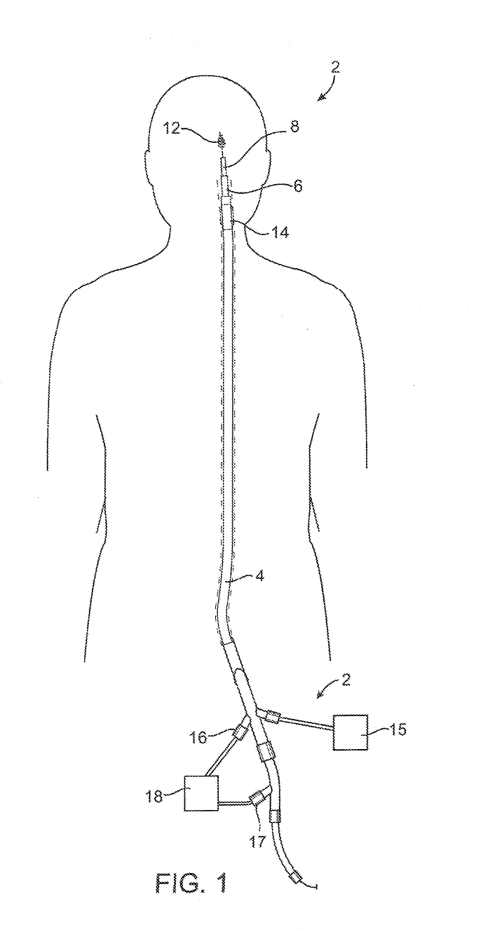

Referring to FIG. 1, a system 2 for removing an obstruction from a vascular location is shown. The system 2 is particularly useful for removing obstructions from the cerebral vasculature. The system 2 includes a guide catheter 4, a support catheter 6, a microcatheter 8, a guidewire and an obstruction retriever 12. The guide catheter 4 is advanced within the vasculature and guides the other catheters and devices through the larger vessels leading to the obstruction. The guide catheter 4 may have a balloon 14 which is coupled to a source of inflation fluid 15 and is inflated during the procedure to temporarily stop blood flow when removing the obstruction. The guide catheter 4 may have a lumen 16 and the support catheter 6 may have a lumen 17 which both may be coupled to a vacuum source 18 for aspirating the obstruction. Of course, the present invention may be practiced with one or more of the catheters without departing from various aspects of the invention. For example, the support catheter 6 could be used without the guide catheter 4 and/or microcatheter 8.

The guidewire is advanced into or distal to the obstruction and the microcatheter 8 is then advanced over the guidewire so that the microcatheter 8 is also positioned within or distal to the obstruction. The guidewire is then withdrawn and the retriever 12 is advanced through the microcatheter 8. The microcatheter 8 is then withdrawn to expose part of the retriever 12 within or distal to the obstruction and withdrawn, if necessary, to contact the obstruction. The retriever 12 may be any suitable device that engages and dislodges the obstruction. For example, the retriever 12 may have a self-expanding element 20 having a free end 22 so that the element 20 is essentially a single filament or wire. The system of the present invention is particularly useful when using small devices to engage and remove the obstruction. In particular, the system of the present invention is useful for retrievers 12 having an outer diameter of less than 0.021 inch and even less than 0.018 inch when collapsed and contained within the microcatheter 8. A suitable device that may be used for the retriever 12 is sold by Concentric Medical.

The support catheter 6 is sized to be advanced through the guide catheter 4 and over the retriever 12 and optionally over the microcatheter 8 as well. The support catheter 6 has a proximal portion, which may be somewhat stiff and rigid for optimized advancement and support. A distal portion of the support catheter 6 is relatively flexible to navigate the tortuous vessels such as the distal ICA and the siphon. Although the support catheter 6 must be flexible enough to navigate these vessels, the support catheter 6 must also still be strong enough to prevent kinking and/or collapse during aspiration.

The support catheter 6 is sized to extend through the guide catheter 4 while optionally being large enough to accommodate the microcatheter 8. The support catheter 6 has a lumen 24 with a diameter of less than 0.065 inch along a distal portion 26 which extends at least 10 cm from a distal end 28. The microcatheter 8 may have an outer diameter of less than 0.060 inch, or even less than 0.050 inch, and may be within a range of about 0.025-0.060 inch along a distal portion 32 which extends for a length of at least 10 cm from a distal end 34. The lumen 17 of the microcatheter 8 may have a diameter of about 0.017 inch while the lumen of the support catheter 6 may have a diameter of about 0.040-0.065 inch.

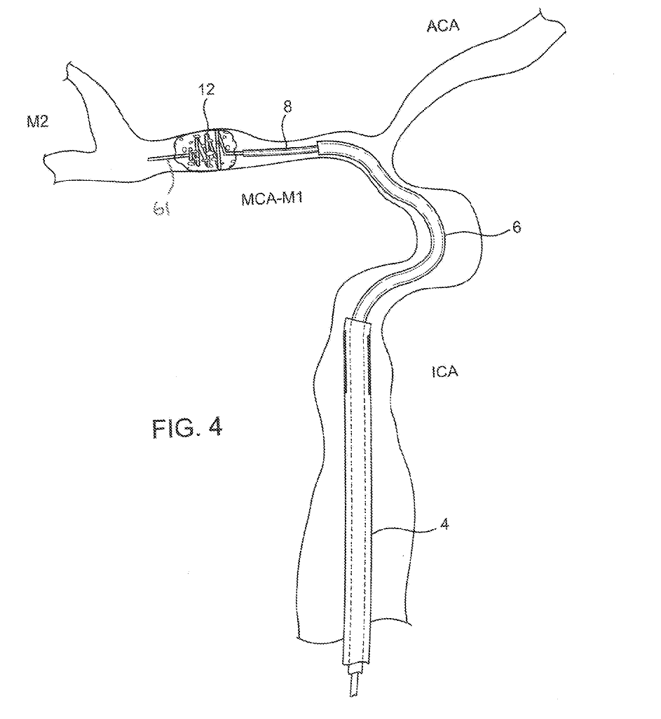

The support catheter 6 is advanced into small, torutuous vessels to provide support when dislodging and removing the obstruction with the retriever 12. The support catheter 6 is advanced further into the vasculature for obstructions in the MCA than typical guide catheters that are too stiff to navigate the distal ICA and siphon. By advancing the support catheter 6 nearer to the clot and retriever 12 than would be possible with typical guide catheters, the problems concerning vessel compression and collapse discussed above may be reduced or avoided. FIG. 4 shows the distal end 28 of the support catheter 6 advanced to a position near the retriever 12. Positioning the support catheter 6 near the retriever 12 may result in a more longitudinally directed force on the obstruction and may also reduce the problem of vessel compression and collapse compared to a typical guide catheter which cannot navigate the distal ICA and siphon.

The guide catheter 4 may be introduced into a femoral artery or other suitable access point. For example, the guide catheter 4 may be an 8Fr catheter with the balloon 14 being used to stop or reduce flow. The microcatheter 8 may be sized small enough to fit within the lumen 24 of the support catheter 6 or may be removed before introduction of the support catheter 6 if the microcatheter 8 is too large to be received by the support catheter 6. Of course, other guide catheters and microcatheters, including those without a balloon, may be used with the systems and methods of the present invention.

The design of the support catheter 6 is now described. The support catheter 6 may have a constant diameter or may have a tapered diameter along one or more sections. The following is a description of a support catheter 6 having a constant diameter. The support catheter 6 has an ID of about 0.060 inch and an OD of about 0.078 inch. The support catheter 6 has a body 40 having a distal tip formed of 25 D pebax and having a length of 1 mm to provide a flexible, atraumatic tip 41. A marker band is provided proximal to the tip 41 and is made of a material, such as platinum, which is readily visible to help identify the distal end of the catheter 6 when viewing the catheter 6 as is known in the art.

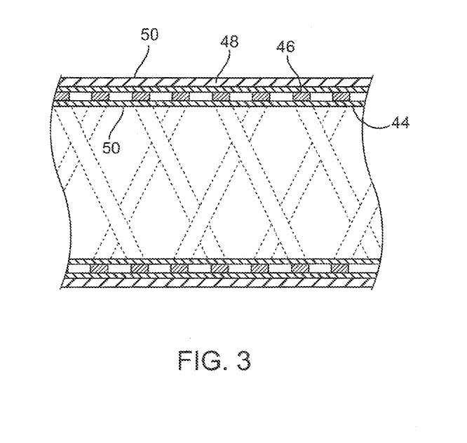

The catheter body 40 has increasing flexibility toward the distal end 28 of the catheter 6. Referring to FIG. 3, the catheter body 40 has a liner 44, a reinforcing layer 46 over the liner 44, an outer tube 48 and a coating 50 on the inner and/or outer surfaces. The liner 44, reinforcing layer 46 and outer tube 48 are fused together using a shrink tube (not shown) as is known in the art. The coating 50 may be a hydrophyllic coating used to reduce friction so that the various catheters and devices described herein may move smoothly against one another and within the vasculature.

The liner 44 may be an etched PTFE liner having a 0.060 inch ID and 0.0015 inch wall thickness. The reinforcing layer 46 may be a braided reinforcing layer made of stainless steel ribbon (0.001×0.005 inch) at 90 PIC. The outer tube 48 has a first section having a length of 15 cm from the marker and has a durometer of 25 D. The outer tube 48 also has a second section having a length of 5 cm and a durometer of 40 D, a third section having a length of 5 cm and a durometer of 55 D and a fourth section having a length of 35 cm and a durometer of 72 D with each section extending from the other toward the proximal end. The support catheter 6 may have additional increases in durometer toward the proximal end. Of course, the increases in stiffness may be accomplished in any other manner such as changing the reinforcing layer 46 to change the stiffness rather than changing the durometer of the outer tube 48.

The catheter 6 has a flexible distal portion 52 which is at least 10 cm long and even at least 15 cm long. The flexible distal portion 52 is designed to navigate the tight bends and tortuousity of the cerebral vessels and, in particular, to traverse the distal ICA and the siphon to reach the distal MCA while maintaining structural integrity throughout the procedure. By advancing the support catheter 6 nearer to the obstruction and the retriever 12 than would be possible with typical catheters, the problems concerning vessel compression and collapse discussed above may be reduced or avoided since the support catheter 6 will provide a more axially directed force relative to the vessel. Another advantage of positioning the support catheter 6 just proximal to the obstruction is that suction can be applied to the lumen of the support catheter 6 to assist in removal of the obstruction.

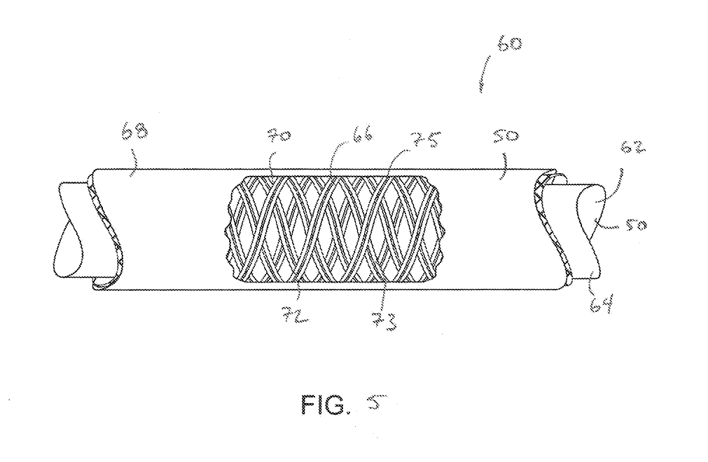

Referring to FIG. 5, a partial cross-sectional view of another support catheter 60 is shown. The support catheter 60 may be used in any manner described herein and may find other uses as well without departing from the scope of the invention. The support catheter 60 provides intravascular support for various catheters in the same manner that it provides support for the microcatheter 8 described above and, as such, provides the same benefits for other types of catheters as well. Such uses are expressly incorporated here as well as the discussion above concerning uses of the support catheter 60 in conjunction with the microcatheter 8 and guide catheter 4 (see FIG. 4). To this end, the obstruction retriever 12 may simply be a working catheter 61 which may be any suitable catheter such as a stent delivery catheter, an embolic coil delivery catheter or any other therapeutic or diagnostic catheter. For example, the support catheter 60 may be used to deliver stents and stent delivery catheters, as well as embolic agents and embolic delivery catheters to the cerebral vasculature without departing from the scope of the invention. The advantages of using the support catheters 60 described herein to deliver these devices is that the support catheters 60 of the present invention offer superior support distal to the guide catheter 4 (see FIG. 4). This support aides in the navigability of these systems during the required manipulation of these system. The support catheters 60 of the present invention are unique in that they are flexible and soft enough to navigate the tortuosity of the internal carotid and the vertebral arteries, allowing delivery to the intracranial circulation, yet have sufficient support properties to facilitate procedures performed more distally. The unique support is at least partially due to the hoop strength on the catheter 60, in part a result of the braid geometry described in further detail below.

The added support not only facilitates these neurointerventional procedures, it also makes them safer. For example, the microcatheter 8 (see FIG. 4) may be used to embolize glues and solidify polymers in arteriovenous malformations (AVM). These microcatheters 8 can frequently become glued into the vessel during the delivery and reflux of the glue. When they are retracted, it can cause vessel stretching and deflection, which can result in vessel dissection or perforation. By using the support catheters 60 of the present invention, the vasculature can be stabilized during microcatheter 8 retraction, thus preventing vessel damage. Another example is the delivery of intracranial stent catheters. These catheter systems can be very bulky and stiff, unable to navigate the carotid siphon or other tortuous vessels. By placing the support catheter 60 distal to the siphon, the relatively bulky delivery catheters now have a direct conduit to the treatment location.

The catheter 60 may have an inner lumen 62 having a diameter of less than 0.040 inch and often within the range of 0.020 to 0.039 inch. Of course, other applications may call for larger sizes without departing from various aspects of the present invention. The inner lumen of the catheter 60 may also be less than 0.020 in, and in the range of 0.010 in to 0.020 in, typically 0.017 in. Also, the catheter may have a diameter greater than 0.020 in, in the range of 0.020 in to 0.070 in, typically 0.045 in or 0.057 in.

The catheter 60 has a liner 64, a reinforcing layer 66 over the liner 64, an outer tube 68 over the reinforcing layer 66 and a coating 50 on the inner and/or outer surfaces. The liner 64, reinforcing layer 66 and outer tube 68 are fused together with a shrink tube (not shown) as is known in the art. The reinforcing layer 66 may formed of any suitable element such as a strand 70 of stainless steel such as 304V SS (325 kpsi +/−40 kpsi. The dimensions of the wire strands are typically 0.0005 in to 0.001 in thick×0.0020 in to 0.0040 in wide, and commonly 0.0007 in×0.0030 in. Nitinol may also be used instead of Stainless Steel.

The reinforcing layer 66 may be a braid 72 such as a diamond braid 73. The braid 72 may be formed with at least twelve strands 70 or even at least sixteen strands 70. The strands 70 may be paired together to form pairs of strands 75 with the strands 70 forming the pair 75 extending substantially parallel to one another. In one embodiment, the diamond braid 72 is formed with a pair of strands 70 being woven over another pair of strands 75 (wound in the opposite direction) and then under two other strands 70 (also wound in the opposite direction) to form the braid 72. The strands are wound to have a braid density of 30-70 PIC (per inch count) and may be about 50 PIC.

The catheter 60 may also have increased flexibility toward the distal end similar to other embodiments described herein. To this end, the outer tube 68 may have a durometer, which increases toward the proximal end as described above in connection with FIG. 3. For example, the outer tube 68 may have a durometer, which varies from 25 D at the distal tip to 80 D at the proximal end of the shaft so that the catheter 60 has increased flexibility toward the distal end.

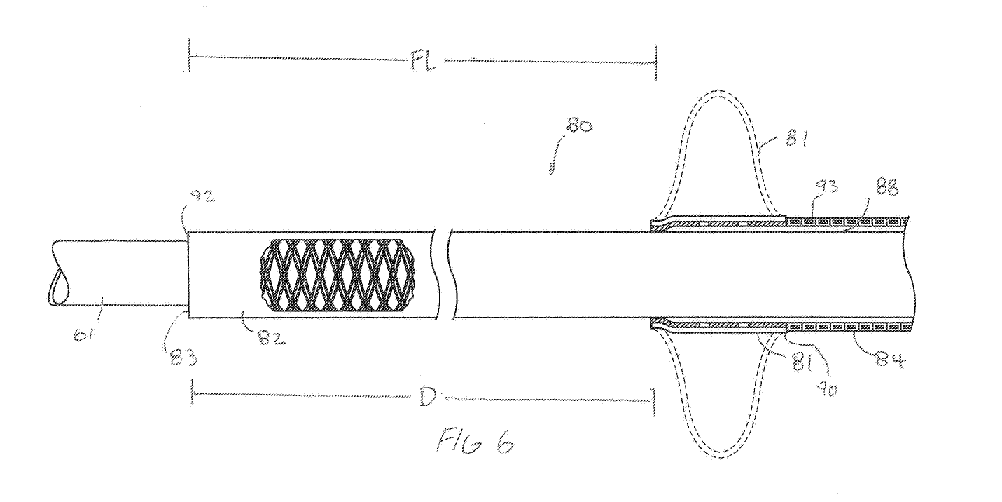

Referring to FIG. 6, still another support catheter 80 is shown which may be used in any manner as any of the other support catheters described herein and such uses are expressly incorporated here. Furthermore, the dimensions and uses of the other catheters and devices used or described in conjunction with any of the support catheters described herein, such as microcatheters, stent delivery catheters and guide catheters, are also expressly incorporated here.

The support catheter 80 differs from support catheters 6, 60 in that the support catheter includes a balloon 81 which may be used to temporarily stop blood flow as is useful in many procedures including many of those described herein. The support catheter includes a lumen 83 through which other devices may pass such as the working catheter 61. The support catheter 80 also includes an inflation lumen 88 coupled to the balloon 81. The support catheter 80 may be formed in any suitable manner with the lumens 81, 88 being either coaxial or side-by-side designs. In one embodiment, the support catheter 80 may have a coaxial design with an inner tube 82 and an outer tube 84. The inflation lumen 88 is formed in the space between the inner and outer tubes 82, 84. The balloon 86 may be mounted to the outer tube 82 entirely or partially mounted to both the inner and outer tubes 82, 84. For example, the balloon 86 may be mounted in a recess 90 formed in the outer tube 84 in a manner similar the method described in U.S. Pat. No. 6,638,245, which is hereby incorporated by reference.

The inner tube 82 may be constructed in accordance with the method of forming the catheter 60 and the braided reinforcement of catheter 60 is expressly incorporated here. The outer tube 84 may have any suitable construction. For example, the outer tube 84 may be an unreinforced polymer tube made of varying durometer from 40 D to 80 D, for example, and having an inner diameter of 0.074 in. and an outer diameter of 0.080 in. when the inner tube 82 has an inner diameter of 0.057 in. Alternatively, the outer tube 84 may have reinforcing element 93, which may be a helical coil or braid, to form a reinforced polymer outer tube 84. The balloon 81 may be formed of silicone tubing have a 0.066 in ID and a 0.006 in wall thickness (which may also be 0.004 to 0.008 inch) when mounted over the outer tube 84 described above and in accordance with the method described in U.S. Pat. No. 6,638,245. Furthermore, the braided reinforcement may include at least 32 strands forming at least 16 pairs of strands braided in accordance with the braided configuration described above and incorporated here. For example, 8 pairs of strands are wound in one direction and 8 pairs of strands are wound in the opposite direction when 32 strands are used.

The balloon 81 may be positioned somewhat far from a distal end 92 of the support catheter 80. Typical guide catheters and microcatheters with balloons have balloons positioned near the distal end of the catheter. A problem with positioning the balloon close to the distal end of the catheter is that the catheter may be difficult to advance into tortuous anatomy due to the stiffness added by the balloon and inflation lumen. The balloon 81 of the support catheter 80 may be positioned a distance D from the distal end of the catheter 80 and/or inner tube 84 of 5-15 cm. Positioning the balloon 81 in this manner permits a free length FL of the support catheter 80, which is formed of the inner tube 82, to access relatively distal vasculature with a relatively large lumen 83 providing many of the advantages as the support catheters 6, 60 with the added benefit of proximal flow control. The outer tube 84 also has a distal end, which, like the balloon 81, is also positioned 5-15 cm from a distal end of the inner tube 82 and/or the support catheter 80 so that the free length FL of the support catheter 80 consists of only the inner tube 82.

The present invention has been described in connection with preferred embodiments, however, it is understood that numerous modifications may be made without departing from the scope of the invention. For example, the catheter may have a tapered body and the reinforcing element may be a helical wire rather than braid without departing from the scope of the invention.

What is claimed is:

1. A catheter, comprising:
an inner tube having a braided reinforcement, the braided reinforcement having at least 12 strands forming a diamond braid, the strands being paired together to form at least six pairs of strands with each strand in the pair extending parallel to the other strand in the pair, wherein at least three of the pairs of strands are wound in one direction and at least three other pairs of strands being wound in an opposite direction, the inner body having a lumen and a distal end;
an outer tube positioned over the inner tube;
an inflation lumen formed between the inner tube and the outer tube; and
a balloon coupled to at least one of the inner and outer tubes, the balloon also being coupled to the inflation lumen.

2. The catheter of claim 1, wherein:
the braided reinforcement of the inner tube has at least 16 strands forming at least 8 pairs of strands.

3. The catheter of claim 1, wherein:
the braided reinforcement of the inner tube has at least 32 strands forming at least 16 pairs of strands.

4. The catheter of claim 1, wherein:
the braided reinforcement is formed with the pairs of strands being wound with 30-70 PIC.

5. The catheter of claim 1, wherein:
the outer tube is an unreinforced polymer tube.

6. The catheter of claim 1, wherein:
the outer tube is reinforced.

7. The catheter of claim 1, wherein:
the balloon is positioned 5-15 cm from the distal end of the inner tube.

8. The catheter of claim 1, wherein:
the outer tube has a distal end which is 5-15 cm from the distal end of the inner tube.

9. A method of accessing a middle cerebral artery, comprising the steps of:
providing a working catheter, a microcatheter, a support catheter and a guide catheter, the support catheter having a balloon;
positioning a distal end of the guide catheter at a position proximal to the MCA;
advancing the microcatheter through the guide catheter;
advancing the support catheter over the microcatheter so that a distal end of the support catheter is positioned in the middle cerebral artery; and
inflating the balloon on the support catheter to occlude blood flow after the advancing step.

10. The method of claim 9, wherein:
the providing step is carried out with the balloon being positioned 5-15 cm from the distal end of the support catheter.

11. The method of claim 9, further comprising the steps of:
positioning the working catheter within a lumen of the microcatheter; and
withdrawing the microcatheter to expose a portion of the working catheter.

12. A catheter, comprising:
an inner tube having a lumen and a distal end;
an outer tube positioned over the inner tube;
an inflation lumen formed between the inner tube and the outer tube; and
a balloon coupled to at least one of the inner and outer tubes, the balloon also being coupled to the inflation lumen, the balloon being positioned 5-15 cm from a distal end of the outer tube.

13. The catheter of claim 12, wherein:
the inner tube has a braided reinforcement, the braided reinforcement having at least 12 strands forming a diamond braid, the strands being paired together to form six pairs of strands with each strand in the pair extending parallel to the other strand in the pair, wherein at least three of the pairs of strands are wound in one direction and at least three other pairs of strands being wound in an opposite direction, the inner body having a lumen and a distal end.

14. The catheter of claim 12, wherein:
the braided reinforcement of the inner tube has at least 16 strands forming at least 8 pairs of strands.

15. The catheter of claim 12, wherein:
the braided reinforcement is formed with the pairs of strands being wound with 30-70 PIC.

16. The catheter of claim 12, wherein:
the outer tube is an unreinforced polymer tube.

17. The catheter of claim 12, wherein:
the outer tube has a distal end which is 5-15 cm from the distal end of the inner tube.

18. The catheter of claim 12, wherein:
the braided reinforcement of the inner tube has at least 32 strands forming at least 16 pairs of strands.

19. The catheter of claim 12, wherein:
the braided reinforcement is formed with the pairs of strands being wound with 30-70 PIC.

20. The catheter of claim 12, wherein:
the outer tube is a reinforced polymer tube.

21. The catheter of claim 12, wherein:
the braided reinforcement of the inner tube has 32 strands with 8 pairs of strands wound in one direction and 8 pairs of strands wound in the opposite direction.
